SEQUENCE LISTING (14 sequences)

SEQ ID NO: 1 - protein, 44 aa, Artificial sequence
  Modified GRF peptide
  MISC_FEATURE (1)..(1): Tyr residue is linked to an hexenoyl-trans-3 moiety
  MOD_RES (44)..(44): AMIDATION
YADAIFTNSYRKVLGQLSARKLLQDIMSRQQGESNQERGARARL

SEQ ID NO: 2 - protein, 44 aa, Artificial sequence
  GRF peptide
  VARIANT (1)..(1): Xaa = Tyr or His
  VARIANT (2)..(2): Xaa = Val or Ala
  VARIANT (8)..(8): Xaa = Asn or Ser
  VARIANT (13)..(13): Xaa = Val or Ile
  VARIANT (15)..(15): Xaa = Ala or Gly
  VARIANT (18)..(18): Xaa = Ser or Tyr
  VARIANT (24)..(24): Xaa = Gln or His
  VARIANT (25)..(25): Xaa = Asp or Glu
  VARIANT (27)..(27): Xaa = Met or Ile or Nle
  VARIANT (28)..(28): Xaa = Ser or Asn
  VARIANT (30)..(30): Xaa = any amino acid or is absent
  VARIANT (31)..(31): Xaa = any amino acid or is absent
  misc_feature (32)..(32): Xaa can be any naturally occurring amino acid
  VARIANT (33)..(33): Xaa = any amino acid or is absent
  VARIANT (34)..(34): Xaa = any amino acid or is absent
  VARIANT (35)..(35): Xaa = any amino acid or is absent
  VARIANT (36)..(36): Xaa = any amino acid or is absent
  VARIANT (37)..(37): Xaa = any amino acid or is absent
  VARIANT (38)..(38): Xaa = any amino acid or is absent
  VARIANT (39)..(39): Xaa = any amino acid or is absent
  VARIANT (40)..(40): Xaa = any amino acid or is absent
  VARIANT (41)..(41): Xaa = any amino acid or is absent
  VARIANT (42)..(42): Xaa = any amino acid or is absent
  VARIANT (43)..(43): Xaa = any amino acid or is absent
  VARIANT (44)..(44): Xaa = any amino acid or is absent
XXDAIFYXSYRKXLXQLXARKLLXXIXXRXXXXXXXXXXXXXXX

SEQ ID NO: 3 - protein, 15 aa, Artificial sequence
  Amino acid sequence corresponding to positions 30 to 44 of the human GRF
QQGESNQERGARARL

SEQ ID NO: 4 - protein, 44 aa, Homo sapiens; identical to SEQ ID NO: 1

SEQ ID NO: 5 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 6 - protein, 26 aa, Homo sapiens
MNFLLSWVHWSLALLLYLHHAKWSQA

SEQ ID NO: 7 - protein, 206 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 8 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 9 - protein, 29 aa, Homo sapiens
MPPSGLRLLLLLLPLLWLLVLTPGRPAAG

SEQ ID NO: 10 - protein, 249 aa, Homo sapiens
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRR

SEQ ID NO: 11 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 12 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSFSPQLQESVFHLLVPSVILVLLAVGGLLFYRWRRRSHQEPQRADSPLEQPEGSPLTQDDRQVELPV

SEQ ID NO: 13 - protein, 32 aa, Homo sapiens
MTAPGAAGRCPPTTWLGSLLLLVCLLASRSIT

SEQ ID NO: 14 - protein, 522 aa, Homo sapiens
EEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSFSPQLQESVFHLLVPSVILVLLAVGGLLFYRWRRRSHQEPQRADSPLEQPEGSPLTQDDRQVELPV

(19) United States
(12) Patent Application Publication
(10) Pub. No.: US 2023/0393148 A1
(43) Pub. Date: Dec. 7, 2023

(54) KITS, REAGENTS AND METHODS FOR THE ASSESSMENT OF LIVER DISEASES

(71) Applicant: The General Hospital Corporation, Boston, MA (US)

(72) Inventor: Steven K. Grinspoon, Weston, MA (US)

(73) Assignee: The General Hospital Corporation, Boston, MA (US)

(21) Appl. No.: 18/034,329

(22) PCT Filed: Oct. 26, 2021
(86) PCT No.: PCT/US2021/056568

§ 371 Date: Apr. 27, 2023

Related U.S. Application Data
(60) Provisional application No. 63/107,730, filed on Oct. 30, 2020.

(51) Int. Cl. G01N 33/68; G01N 33/53; A61K 38/25; A61P 1/16

(52) U.S. Cl. CPC G01N 33/6893; G01N 33/5308; A61K 38/25; A61P 1/16; G01N 2333/495; G01N 2333/53; G01N 2800/56

(57) ABSTRACT

The present application relates to methods, reagents and kits for the assessment of the severity of nonalcoholic fatty liver disease (NAFLD) in a patient over time, and/or of the likelihood that a subject suffers from NAFLD. The methods, reagents and kits are based on the determination of the levels of VEGFA, TGFB1, and/or CSF1 in a biological sample, such as a plasma sample, from the subject. The methods, reagents and kits may be used, for example, for assessing the progression of NAFLD in a patient and/or the response of the patient to therapy.

### CROSS REFERENCE TO RELATED APPLICATIONS

The present application claims the benefit of U.S. Provisional Application Ser. No. 63/107,730, filed on Oct. 30, 2020, which is incorporated herein by reference in its entirety.

### SEQUENCE LISTING

This application contains a Sequence Listing in computer readable form entitled “11718_419_SeqList.txt”, created on Oct. 1, 2021 and having a size of about 25 kB. The computer readable form is incorporated herein by reference.

### TECHNICAL FIELD

The present disclosure generally relates to the field of liver diseases, and more particularly to the assessment of the status and progression of nonalcoholic fatty liver (NAFL), nonalcoholic steatohepatitis (NASH), and liver fibrosis.

### BACKGROUND ART

NAFL is defined by excess storage of triglyceride in hepatocytes (steatosis) and is often characterized by resultant inflammation, cellular ballooning and damage, and fibrosis. Significant changes in this regard lead to NASH. Nonalcoholic fatty liver disease (NAFLD) may progress to fibrosis and ultimately cirrhosis and is an increasingly important cause of end-stage liver disease in the general population, and has also been studied in people living with HIV (1-6). NAFL/NASH have a higher prevalence in HIV patients and tend to progress faster than in the general population. In contrast to many HIV-associated comorbidities that worsen with increased HIV-disease severity, NAFLD may occur more commonly in HIV patients with weight gain, and it is associated with central adiposity. In people living with HIV (PLWH), weight gain, abdominal fat accumulation, and increases in visceral fat are common and seen even with newer antiretrovirals.

There are currently no simple and reliable assays to monitor NAFLD/NASH development and progression in a patient. The presence of NASH is the main predictor of development and progression to liver fibrosis, and progression of liver fibrosis is the main determinant of adverse liver-related clinical outcomes. Therefore, identifying and monitoring NAFLD/NASH and advanced fibrosis have important prognostic and disease management implications.

NAFLD/NASH may be suspected in subjects with increased levels of the liver enzymes alanine aminotransferase (ALT) and aspartate aminotransferase (AST), but these markers are also upregulated in other liver conditions. Imaging techniques such as ultrasound, computerized tomography (CT) scans, magnetic resonance imaging (MRI), ultrasound elastography (USE), quantitative ultrasound-based techniques, magnetic resonance elastography (MRE), and magnetic resonance-based fat quantitation technique, are also used to detect fat in the liver, but they usually fail to detect liver inflammation and/or fibrosis. Also, these techniques require specialized imaging devices and analysis of the images by a radiologist. Liver biopsy remains the gold standard for the diagnosis and staging of NASH, mainly due to the lack of a reliable noninvasive method. However, liver biopsy is expensive, subjective, and associated with risks for patients.

There is thus a need for the development of simple, reliable non-invasive assays for the assessment of the status and progression of NAFLD/NASH and liver fibrosis in patients.

The present description refers to a number of documents, the content of which is herein incorporated by reference in their entirety.

### SUMMARY OF THE DISCLOSURE

The present disclosure generally relates to the field of liver diseases, and more particularly to the assessment of the status and progression of nonalcoholic fatty liver (NAFL), nonalcoholic steatohepatitis (NASH), and liver fibrosis.

In various aspects and embodiments, the present disclosure provides the following items:

1. A method for assessing the severity of nonalcoholic fatty liver disease (NAFLD) in a patient over time, the method comprising
measuring a first protein level of Vascular Endothelial Growth Factor A (VEGFA), Transforming Growth Factor Beta 1 (TGFB1), and/or Colony Stimulating Factor 1 (CSF1) in a biological sample from the patient at a first time point;
measuring a second protein level of VEGFA, TGFB1, and/or CSF1 in a corresponding biological sample from the patient at a second, later time point;
   wherein a decrease in the second protein level relative to the first protein level of VEGFA, TGFB1, and/or CSF1 between said first and second time points is indicative that NAFLD severity has regressed over time in the patient;
   wherein an increase in the second protein level relative to the first protein level of VEGFA, TGFB1, and/or CSF1 between said first and second time points is indicative that NAFLD severity has progressed over time in the patient; and
   wherein no change in the second protein level relative to the first protein level of VEGFA, TGFB1, and CSF1 between said first and second time points is indicative that NAFLD severity has been stable over time in the patient.
   2. The method of item 1, wherein the method comprises measuring protein levels of VEGFA.
   3. The method of item 1 or 2, wherein the method comprises measuring protein levels of TGFB1.
   4. The method of any one of items 1 to 3, wherein the method comprises measuring protein levels of CSF1.
   5. The method of any one of items 1 to 4, wherein NAFLD severity comprises the fibrosis score and/or the NAFLD Activity Score (NAS).
   6. The method of item 5, wherein NAFLD severity comprises the fibrosis score.
   7. The method of item 5 or 6, wherein NAFLD severity comprises the NAS.
   8. The method of any one of items 1 to 7, wherein the patient has received a treatment against NAFLD between said first time point and said second time point.
   9. The method of item 8, wherein the treatment comprises administration of a Growth Hormone-Releasing Hormone (GHRH) molecule or an analog thereof.
   10. The method of item 9, wherein the treatment comprises administration of trans-3-hexenoyl-GHRH_{(1.44)}-NH₂ or a pharmaceutically acceptable salt thereof.
   11. The method of any one of items 1 to 10, wherein the biological sample is a blood-derived sample.
   12. The method of item 11, wherein the blood-derived sample is plasma.
   13. The method of any one of items 1 to 12, wherein measuring protein levels of VEGFA comprises contacting the biological sample with an antibody or antigen-binding fragment thereof that specifically binds to VEGFA, and measuring the amount of complexes between VEGFA and the antibody or antigen-binding fragment thereof.
   14. The method of any one of items 1 to 13, wherein measuring protein levels of TGFB1 comprises contacting the biological sample with an antibody or antigen-binding fragment thereof that specifically binds to TGFB1, and measuring the amount of complexes between TGFB1 and the antibody or antigen-binding fragment thereof.
   15. The method of any one of items 1 to 14, wherein measuring protein levels of CSF1 comprises contacting the biological sample with an antibody or antigen-binding fragment thereof that specifically binds to CSF1, and measuring the amount of complexes between CSF1 and the antibody or antigen-binding fragment thereof.
   16. The method of any one of items 13 to 15, wherein the antibody or antigen-binding fragment thereof is conjugated to a detectable moiety.
   17. The method of any one of items 1 to 16, wherein said patient suffers from human immunodeficiency virus (HIV) infection.
   18. A method for assessing the likelihood that a subject suffers from nonalcoholic fatty liver disease (NAFLD), the method comprising measuring protein levels of Vascular Endothelial Growth Factor A (VEGFA), Transforming Growth Factor Beta 1 (TGFB1), and/or Colony Stimulating Factor 1 (CSF1) in a biological sample from the subject, wherein a higher level of VEGFA, TGFB1, and/or CSF1 in the sample relative to a corresponding control level is indicative of an increased likelihood that the subject suffers from NAFLD.
   19. The method of item 18, wherein the method comprises measuring protein levels of VEGFA.
   20. The method of item 18 or 19, wherein the method comprises measuring protein levels of TGFB1.
   21. The method of any one of items 18 to 20, wherein the method comprises measuring protein levels of CSF1.
   22. The method of any one of items 18 to 21, wherein the NAFLD is nonalcoholic steatohepatitis (NASH).
   23. The method of any one of items 18 to 22, wherein the NAFLD comprises liver fibrosis.
   24. The method of any one of items 18 to 23, wherein the biological sample is a blood-derived sample.
   25. The method of item 23, wherein the blood-derived sample is plasma.
   26. The method of any one of items 18 to 25, wherein measuring protein levels of VEGFA comprises contacting the biological sample with an antibody or antigen-binding fragment thereof that specifically binds to VEGFA, and measuring the amount of complexes between VEGFA and the antibody or antigen-binding fragment thereof.
   27. The method of any one of items 18 to 26, wherein measuring protein levels of TGFB1 comprises contacting the biological sample with an antibody or antigen-binding fragment thereof that specifically binds to TGFB1, and measuring the amount of complexes between TGFB1 and the antibody or antigen-binding fragment thereof.
   28. The method of any one of items 18 to 27, wherein measuring protein levels of CSF1 comprises contacting the biological sample with an antibody or antigen-binding fragment thereof that specifically binds to CSF1, and measuring the amount of complexes between CSF1 and the antibody or antigen-binding fragment thereof.
   29. The method of any one of items 26 to 28, wherein the antibody or antigen-binding fragment thereof is conjugated to a detectable moiety.
   30. The method of any one of items 18 to 29, wherein the method is performed on a biological sample from a subject that is suspected of suffering from NAFLD.
   31. The method of item 30, wherein the subject has increased levels of alanine aminotransferase (ALT) and/or aspartate aminotransferase (AST).
   32. The method of any one of items 18 to 31, wherein said subject suffers from human immunodeficiency virus (HIV) infection.
   33. A method for treating nonalcoholic fatty liver disease (NAFLD), the method comprising identifying a subject having an increased likelihood of suffering from NAFLD using the method of any one of items 18 to 32, and administering a treatment against NAFLD to the subject.
   34. The method of item 33, wherein the treatment comprises administration of Growth Hormone-Releasing Hormone (GHRH) or an analog thereof.
   35. The method of item 34, wherein the treatment comprises administration of trans-3-hexenoyl-GHRH₍₁₋₄₄₎-NH₂ or a pharmaceutically acceptable salt thereof.
   36. A kit for use in (a) assessing the severity of nonalcoholic fatty liver disease (NAFLD) in a patient over time, and/or (b) assessing the likelihood that a subject suffers from NAFLD, the kit comprising reagents for measuring protein levels of Vascular Endothelial Growth Factor A (VEGFA), Transforming Growth Factor Beta 1 (TGFB1), and/or Colony Stimulating Factor 1 (CSF1) in a biological sample; and instructions for correlating the protein levels of VEGFA, TGFB1, and/or CSF1 with the severity of NAFLD and/or the likelihood of suffering from NAFLD.
   37. The kit of item 36, wherein the kit comprises (i) an antibody or antigen-binding fragment thereof that specifically binds to VEGFA, (ii) an antibody or antigen-binding fragment thereof that specifically binds to TGFB1; (iii) an antibody or antigen-binding fragment thereof that specifically binds to CSF1; or (iv) any combination of (i) to (iii).
   38. The kit of item 37, wherein the antibody or antigen-binding fragment thereof is conjugated to a detectable moiety.
   39. An assay mixture comprising (a) reagents for measuring protein levels of Vascular Endothelial Growth Factor A (VEGFA), Transforming Growth Factor Beta 1 (TGFB1), and/or Colony Stimulating Factor 1 (CSF1) in a biological sample; and (b) a biological sample from a subject suffering from, or suspected of suffering from, nonalcoholic fatty liver disease (NAFLD).
   40. The assay mixture of item 39, wherein the assay mixture comprises (i) an antibody or antigen-binding fragment thereof that specifically binds to VEGFA, (ii) an antibody or antigen-binding fragment thereof that specifically binds to TGFB1; (iii) an antibody or antigen-binding fragment thereof that specifically binds to CSF1; or (iv) any combination of (i) to (iii).
   41. The assay mixture of item 40, wherein the assay mixture comprises any combination of (i) to (iii).
   42. The assay mixture of item 40 or 41, wherein the antibody or antigen-binding fragment thereof is conjugated to a detectable moiety.
   43. The assay mixture of any one of items 39 to 42, wherein the biological sample is a blood-derived sample.
   44. The assay mixture of item 43, wherein the blood-derived sample is plasma.
   45. The assay mixture of any one of items 39 to 44, wherein the biological sample is from a subject suffering from NAFLD.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 is a schematic of the analysis performed in the studies described herein. A total of 13 plasma proteins were examined, which corresponded to top leading-edge genes within differentially modulated hepatic gene pathways. The analysis was focused on the subset of 9 proteins in which the directionality of treatment effect was concordant with the directionality of change in hepatic gene expression. Abbreviations: CASP8, caspase 8; CCL20, C-C motif chemokine ligand 20; CRTAM, cytotoxic and regulatory T-cell molecule; CSF1, macrophage colony stimulating factor 1; CXCL12, C-X-C motif chemokine ligand 12; NCR1, natural cytotoxicity triggering receptor 1; TGFB1, transforming growth factor beta 1; TNFRSF21, tumor necrosis factor receptor superfamily member 21; VEGFA, vascular endothelial growth factor A.

FIGS. 2A-C are graphs showing the differential changes in plasma VEGFA (FIG. 2A), TGFB1 (FIG. 2B), and CSF1 (FIG. 2C) by treatment status. Tesamorelin led to significant reductions in plasma VEGFA (log₂-fold change, mean±SD, −0.20±0.35 vs. 0.05±0.34, P=0.02), TGFB1 (log₂-fold change −0.35±0.56 vs. −0.05±0.43, P=0.05), and CSF1 (log₂-fold change −0.17±0.21 vs. 0.02±0.20, P=0.004) relative to placebo. Bars and error bars indicate mean and standard error of the mean, respectively. Abbreviations: CSF1, macrophage colony stimulating factor 1; TGFB1, transforming growth factor beta 1; VEGFA, vascular endothelial growth factor A.

FIGS. 3A and B are graphs showing the relationship of changes in Plasma VEGFA (FIG. 3A) and CSF1 (FIG. 3B) with change in NAS score in tesamorelin-treated participants. Within the tesamorelin-treated arm, reductions in plasma VEGFA (r=0.62, P=0.006) and CSF1 (r=0.50, P=0.04) were associated with a decrease in NAS score. Linear regression lines with 95% confidence intervals are shown. Abbreviations: CSF1, macrophage colony stimulating factor 1; NAS, NAFLD activity score; VEGFA, vascular endothelial growth factor A.

FIGS. 4A and 4B are graphs depicting the relationship of changes in plasma TGFB1 and CSF1 with change in gene-level fibrosis score. Among tesamorelin-treated participants, declines in plasma TGFB1 (FIG. 4A) (r=0.61, P=0.009) and CSF1 (FIG. 4B) (r=0.64, P=0.006) were associated with improved gene-level fibrosis score. Linear regression lines with 95% confidence intervals are shown. Abbreviations: CSF1, macrophage colony stimulating factor 1; TGFB1, transforming growth factor beta 1.

FIG. 5 shows the amino acid sequence of human VEGFA (SEQ ID NO: 5). Amino acids 1-26 (SEQ ID NO: 6) define the signal peptide; amino acids 27-232 (SEQ ID NO: 7) define the mature polypeptide.

FIG. 6 shows the amino acid sequence of human TGFB1 (SEQ ID NO: 8). Amino acids 1-29 (SEQ ID NO: 9) define the signal peptide; amino acids 30-278 (SEQ ID NO: 10) define the latency-associated peptide; amino acids 279-390 (SEQ ID NO: 11) define the mature polypeptide.

FIG. 7 shows the amino acid sequence of human CSF1 (SEQ ID NO: 12). Amino acids 1-32 (SEQ ID NO: 13) define the signal peptide and residues 33-450 defining the processed mature form (SEQ ID NO: 14).

FIG. 8 shows the structure of tesamorelin (trans-3-hexenoyl-GHRH₍₁₋₄₄₎-NH₂; SEQ ID NO: 1).

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in stem cell biology, cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

In the studies described herein, the present inventors have shown that reduced levels of Vascular Endothelial Growth Factor A (VEGFA), Transforming Growth Factor Beta 1 (TGFB1), and Colony Stimulating Factor 1 (CSF1) are detected in the plasma of patients suffering from NAFLD treated with tesamorelin. The reduction in VEGFA, TGFB1, and/or CSF1 levels were shown to correlate with improvements of pathological features of NAFLD, such as a reduction of the NAFLD Activity Score (NAS) and/or gene-level fibrosis score in the patients.

NAFLD Activity Score (NAS) calculated according to the NAS Clinical Research Network (NAS CRN) scoring system comprises the sum of grades for steatosis (grades 0-3), hepatocellular ballooning (grades 0-2), and lobular inflammation (grades 0-3) (Kleiner D E, et al. Hepatology 2005; 41:1313-21).

In an aspect, the present disclosure provides a method for assessing the likelihood that a subject suffers from NAFLD, the method comprising measuring protein levels of Vascular Endothelial Growth Factor A (VEGFA), Transforming Growth Factor Beta 1 (TGFB1), and/or Colony Stimulating Factor 1 (CSF1) in a biological sample from the subject, wherein a higher level of VEGFA, TGFB1, and/or CSF1 in the sample relative to a corresponding control level is indicative of an increased likelihood that the subject suffers from NAFLD.

“Control level” or “reference level” or “standard level” are used interchangeably herein and broadly refers to a separate baseline level measured in one or more comparable “control” samples, which may be from subjects not suffering from the disease (e.g., NAFLD). The corresponding control level may be a level corresponding to an average/mean or median level calculated based of the levels measured in several reference or control subjects (e.g., a pre-determined or established standard level). The control level may be a pre-determined “cut-off” value recognized in the art or established based on levels measured in samples from one or a group of control subjects. For example, the “threshold reference level” may be a level corresponding to the minimal level of VEGFA, TGFB1, and/or CSF1 (cut-off) that permits to distinguish in a statistically significant manner patients having a higher likelihood or risk of suffering from NAFLD from those not having a higher likelihood or risk of suffering from NAFLD, which may be determined using samples from NAFLD patients and from healthy subjects (i.e., not suffering from NAFLD), for example. The corresponding reference/control level may be adjusted or normalized for age, gender, race, or other parameters. The “control level” can thus be a single number/value, equally applicable to every patient individually, or the control level can vary, according to specific subpopulations of patients. Thus, for example, older men may have a different control level than younger men, and women may have a different control level than men. The predetermined standard level can be arranged, for example, where a tested population is divided equally (or unequally) into groups, such as a low-risk group, a medium-risk group and a high-risk group or into quadrants or quintiles, the lowest quadrant or quintile being individuals with the lowest risk (i.e., lowest levels of VEGFA, TGFB1, and/or CSF1) and the highest quadrant or quintile being individuals with the highest risk (i.e., highest levels of VEGFA, TGFB1, and/or CSF1). It will also be understood that the control levels according to the disclosure may be, in addition to predetermined levels or standards, levels measured in other samples (e.g., from healthy/normal subjects) tested in parallel with the experimental sample. The reference or control levels may correspond to normalized levels, i.e., reference or control values subjected to normalization based on the expression of a housekeeping gene.

In embodiments, the control level is a corresponding level of VEGFA, TGFB1, and/or CSF1 determined in a biological sample of a subject known not to suffer from NAFLD, or an established reference or standard level of VEGFA, TGFB1, and/or CSF1.

The present disclosure also provides a method for assessing the severity of nonalcoholic fatty liver disease (NAFLD) in a patient over time, the method comprising:
measuring protein levels of VEGFA, TGFB1, and/or CSF1 in a biological sample from the patient at a first time point;
measuring protein levels of VEGFA, TGFB1, and/or CSF1 in a corresponding biological sample from the patient at a second, later time point;
   wherein a decrease in protein levels of VEGFA, TGFB1, and/or CSF1 between said first and second time points is indicative that NAFLD severity has regressed over time in the patient;
   wherein an increase in protein levels of VEGFA, TGFB1, and/or CSF1 between said first and second time points is indicative that NAFLD severity has progressed over time in the patient; and
   wherein no change in protein levels of VEGFA, TGFB1, and CSF1 between said first and second time points is indicative that NAFLD severity has been stable over time in the patient.

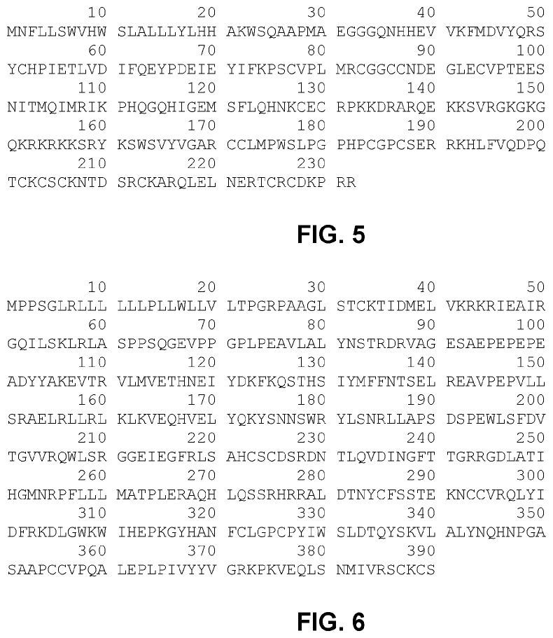

VEGFA (UniProtKB accession No. P15692) is a protein of 232 amino acids (precursor, isoform 1), with amino acids 1-26 defining the signal peptide and amino acids 27-232 defining the mature polypeptide. The amino acid sequence of VEGFA (isoform 1) is depicted at FIG. 5.

TGFB1 (UniProtKB accession No. P01137) is a protein of 390 amino acids (precursor), with amino acids 1-29 defining the signal peptide, and which is proteolytically processed to produce a mature peptide of 112 amino acid (residues 279-390). The amino acid sequence of TGFB1 is depicted at FIG. 6.

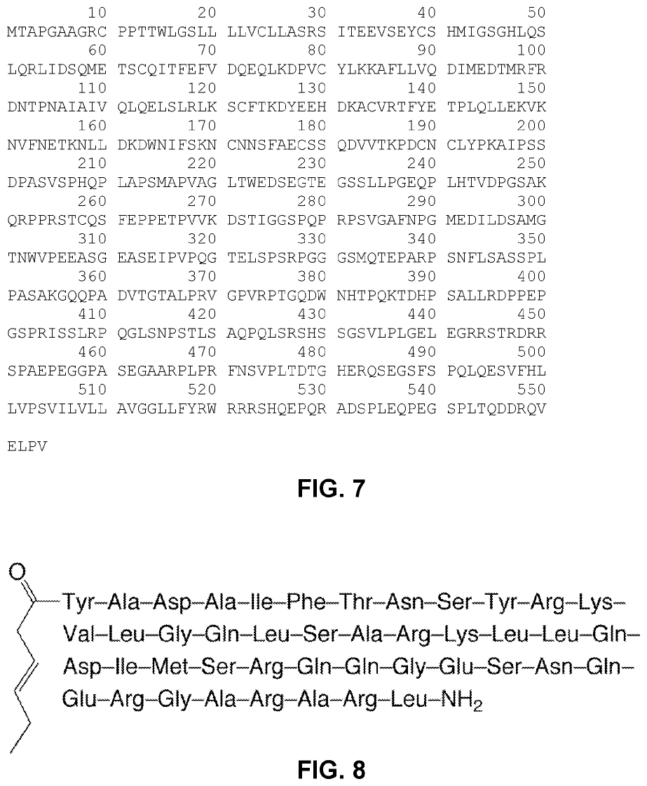

CSF1 (UniProtKB accession No. P09603) is initially produced as a precursor that is membrane bound but processed and secreted upon stimulation. The precursor comprises 554 amino acids (isoform 1), with amino acids 1-32 defining the signal peptide, and residues 33-450 defining the processed mature form. The amino acid sequence of CSF1 (isoform 1) is depicted at FIG. 7.

The above-noted method for assessing the severity of NAFLD over time may be performed at several time points, i.e., protein levels of VEGFA, TGFB1, and/or CSF1 in corresponding biological sample(s) from the patient may be performed at a third, fourth, fifth, etc. time points. The interval between two time points may be, e.g., 1 day, 2 days, 3 days, 1 week, 2 weeks, 1 month, 2 months, 3 months, 6 months, 1 year, etc., and may be the same for all time points or may vary (e.g., 1 week between the first and second time points, and 1 month between the second and third time points).

The method permits to determine whether the patient's condition improves, deteriorates, or is stable over time. In an embodiment, the protein levels of TGFB1 are decreased between a first and a second time point, and the decrease is indicative of a reduction of the NAS score and/or liver fibrosis in the patient. In an embodiment, the protein levels of TGFB1 are increased between a first and a second time point, and the increase is indicative of an increase of the NAS score and/or liver fibrosis in the patient. In an embodiment, the protein levels of CSF1 are decreased between a first and a second time point, and the decrease is indicative of a reduction of the NAS score and/or liver fibrosis in the patient. In an embodiment, the protein levels of CSF1 are increased between a first and a second time point, and the increase is indicative of an increase of the NAS score and/or liver fibrosis in the patient. In an embodiment, the protein levels of VEGFA are decreased between a first and a second time point, and the decrease is indicative of a reduction of the NAS score. In an embodiment, the protein levels of VEGFA are increased between a first and a second time point, and the increase is indicative of an increase of the NAS score. In an embodiment, the protein levels of VEGFA and CSF1 are decreased between a first and a second time point, and the decrease is indicative of a reduction of the NAS score in the patient. In an embodiment, the protein levels of VEGFA and CSF1 are increased between a first and a second time point, and the increase is indicative of an increase of the NAS score in the patient. In an embodiment, the protein levels of TGFB1 and CSF1 are decreased between a first and a second time point, and the decrease is indicative of a reduction of the liver fibrosis in the patient. In an embodiment, the protein levels of TGFB1 and CSF1 are increased between a first and a second time point, and the increase is indicative of an increase of the liver fibrosis in the patient.

The above-noted method for assessing the severity of NAFLD over time may be useful for determining whether a patient suffering from NAFLD responds or not to a treatment/therapy against NAFLD, i.e., to determine whether the treatment/therapy is effective and improves the patient's condition or not. Thus, in another embodiment, the patient is being administered a treatment/therapy between the first and second time points. In another embodiment, the patient undergoes a weight loss program, i.e., healthy (low calorie) diet and/or physical exercise, between the first and second time points.

Accordingly, in another aspect, the present disclosure relates to a method for assessing whether a treatment improves the condition of a patient suffering from NAFLD, the method comprising:
measuring protein levels of VEGFA, TGFB1, and/or CSF1 in a biological sample from the patient at a first time point;
administering a treatment against NAFLD to the patient for a period of time; and
measuring protein levels of VEGFA, TGFB1, and/or CSF1 in a corresponding biological sample from the patient at a second time point after said period of time;
   wherein a decrease in protein levels of VEGFA, TGFB1, and/or CSF1 between said first and second time points is indicative that the treatment has improved the patient's condition;
   wherein no change or an increase in protein levels of VEGFA, TGFB1, and/or CSF1 between said first and second time points is indicative that the treatment has not improved the patient's condition.

In an embodiment, the improvement of the patient's condition comprises reduction of the NAS score. In a further embodiment, the improvement of the patient's condition comprises reduction of the NAS score and the method comprises measuring the levels of VEGFA and/or CSF1.

In an embodiment, the improvement of the patient's condition comprises reduction of liver fibrosis. In a further embodiment, the improvement of the patient's condition comprises reduction of liver fibrosis and the method comprises measuring the levels of TGFB1 and/or CSF1.

In an embodiment, the improvement of the patient's condition comprises reduction of the NAS score and reduction of liver fibrosis. In a further embodiment, the improvement of the patient's condition comprises reduction of the NAS score and reduction of liver fibrosis and the method comprises measuring the levels of CSF1.

In another aspect, the present disclosure relates to a method for determining whether a candidate therapy may be useful for the treatment of NAFLD, the method comprising:
measuring a first protein level of VEGFA, TGFB1, and/or CSF1 in a biological sample from a subject suffering from NAFLD;
administering the candidate therapy to the subject for a period of time; and
measuring a second protein level of VEGFA, TGFB1, and/or CSF1 in a biological sample from the subject after said period of time;
wherein a lower level of the second protein level relative to the first protein level is indicative that the candidate therapy may be useful for the treatment of NAFLD.

In an embodiment, such studies are carried out in the context of a clinical trial that typically entails additionally administering a placebo to a second subject suffering from NAFLD. In such a case, in an embodiment, the method for determining whether a candidate therapy may be useful for the treatment of NAFLD comprises:
measuring first protein levels of VEGFA, TGFB1, and/or CSF1 in biological samples from first and second subjects suffering from NAFLD;
administering the candidate therapy to the first subject and a placebo to the second subject for a period of time; and
measuring second protein levels of VEGFA, TGFB1, and/or CSF1 in biological samples from the first and second subjects after said period of time.

Similarly, in such an embodiment, a decrease in the level of the second protein level relative to the first protein level of VEGFA, TGFB1, and/or CSF1 in the biological sample from the first subject is indicative that the candidate therapy may be useful for the treatment of NAFLD. The determination of the first and second protein levels in the second subject provide an additional control in the context of such a trial.

In an embodiment, the above-mentioned methods comprise measuring protein levels of VEGFA. In an embodiment, the above-mentioned methods comprise measuring protein levels of TGFB1. In an embodiment, the above-mentioned methods comprise measuring protein levels of CSF1. In an embodiment, the above-mentioned methods comprise measuring protein levels of VEGFA and TGFB1. In an embodiment, the above-mentioned methods comprise measuring protein levels of VEGFA and CSF1. In an embodiment, the above-mentioned methods comprise measuring protein levels of TGFB1 and CSF1. In an embodiment, the above-mentioned methods comprise measuring protein levels of VEGFA, TGFB1 and CSF1.

In another aspect, the present disclosure relates to a method for treating nonalcoholic NAFLD, the method comprising administering a treatment against NAFLD to a subject having an increased likelihood of suffering from NAFLD identifying using the method described herein.

In another aspect, the present disclosure relates to a method for treating nonalcoholic NAFLD, the method comprising identifying a subject having an increased likelihood of suffering from NAFLD using the method described herein, and administering a treatment against NAFLD to the subject.

In another aspect, the present disclosure relates to the use of a treatment against NAFLD in a subject, wherein the subject is identified by the method of identifying a subject having an increased likelihood of suffering from NAFLD described herein.

In another aspect, the present disclosure relates to a treatment/therapy for use in a treatment against NAFLD in a subject, wherein the subject is identified by the method of identifying a subject having an increased likelihood of suffering from NAFLD described herein.

The treatment/therapy administered to or performed on the patient in the methods described herein may be an experimental or candidate treatment/therapy, e.g., a treatment/therapy tested in a clinical study, or an approved or established treatment/therapy for NAFLD.

In an embodiment, the treatment/therapy comprises administration or use of a cholesterol-lowering medication, such as statins (e.g., Atorvastatin, Fluvastatin, Lovastatin, Pitavastatin, Pravastatin, Rosuvastatin, Simvastatin), bile acid sequestrants (e.g., Cholestyramine, Colesevelam, Colestipol), cholesterol absorption blockers (e.g., ezetimibe), PCSK9 inhibitors (e.g., anti-PCSK9 antibodies such as Alirocumab and Evolocumab), niacin, fibrates (e.g., Fenofibrate, Gemfibrozil), Adenosine triphosphate-citrate Lyase (ACL) inhibitors (e.g., bempedoic acid), or omega-3 products (e.g., Icosapent ethyl, Omega-3-acid ethyl esters). In another embodiment, the treatment/therapy comprises a change in lifestyle, e.g., undergoing a weight loss program, i.e., healthy (low calorie) diet and/or physical exercise.

In an embodiment, the treatment/therapy comprises administration or use of a GHRH molecule. The term “GHRH molecule” as used in the context of the present disclosure includes, without limitation, human native GHRH₍₁₋₄₄₎ and fragments thereof (e.g., GHRH₍₁₋₄₀₎, GHRH₍₁₋₂₀₎, fragments ranging between 1-29 and the 1-44 sequence), and any other fragments; GHRH from other species and fragments thereof; GHRH variants containing amino acid(s) substitution(s), addition(s) and/or deletion(s); derivatives or analogs of GHRH or fragments or variants thereof having for example an organic group or a moiety coupled to the GHRH amino acid sequence at the N-terminus, the C-terminus or on the side-chain; and pharmaceutically acceptable salts of GHRH (human or from other species), as well as pharmaceutically acceptable salts of native GHRH or fragments, variants, analogs and derivatives thereof. The GHRH molecules of the present disclosure also encompass the GHRH molecules currently known in the art, including, without limitation, albumin-conjugated GHRH (U.S. Pat. No. 7,268,113); pegylated GHRH peptide (U.S. Pat. Nos. 7,256,258 and 6,528,485); porcine GHRH (1-40) (U.S. Pat. No. 6,551,996); canine GHRH (U.S. patent application no. 2005/0064554); GHRH variants of 1-29 to 1-44 amino acid length (U.S. Pat. Nos. 5,846,936, 5,696,089, 5,756,458 and 5,416,073, and U.S. patent application Nos. 2006/0128615 and 2004/0192593); and Pro⁰-GHRHpeptide and variants thereof (U.S. Pat. No. 5,137,872).

The GHRH analogs include those described in U.S. Pat. Nos. 5,681,379 and which also describe their method of synthesis. More particularly, these GHRH analogs are defined by the following formula A:

X-GHRH Peptide (A)
wherein the GHRH peptide is a peptide of the following formula B (SEQ ID NO:2):

A1-A2-Asp-Ala-Ile-Phe-Thr-A8-Ser-Tyr-Arg-Lys-A13-Leu-A15-Gln-Leu-A18-Ala-Arg-Lys-Leu-Leu-A24-A25-Ile-A27-A28-Arg-A30-A31-A32-A33-A34-A35-A36-A37-A38-A39-A40-A41-A42-A43-A44-R0 (B)
wherein,
   A1 is Tyr or His;
   A2 is Val or Ala;
   A8 is Asn or Ser;
   A13 is Val or Ile;
   A15 is Ala or Gly;
   A18 is Ser or Tyr;
   A24 is Gln or His;
   A25 is Asp or Glu;
   A27 is Met, Ile or Nle
   A28 is Ser or Asn;
   A30 is absent or is any amino acid, preferably Gln;
   A31 is absent or is any amino acid, preferably Gln;
   A32 is absent or is any amino acid, preferably Gly;
   A33 is absent or is any amino acid, preferably Glu;
   A34 is absent or is any amino acid, preferably Ser;
   A35 is absent or is any amino acid, preferably Asn;
   A36 is absent or is any amino acid, preferably Gln;
   A37 is absent or is any amino acid, preferably Glu;
   A38 is absent or is any amino acid, preferably Arg;
   A39 is absent or is any amino acid, preferably Gly;
   A40 is absent or is any amino acid, preferably Ala;
   A41 is absent or is any amino acid, preferably Arg;
   A42 is absent or is any amino acid, preferably Ala;
   A43 is absent or is any amino acid, preferably Arg;
   A44 is absent or is any amino acid, preferably Leu; and
   R0 is NH₂ or NH—(CH₂)n—CONH₂, with n=1 to 12.

The group X is a hydrophobic tail anchored via an amide bond to the N-terminus of the peptide and the hydrophobic tail defining a backbone of 5 to 7 atoms. The backbone can be substituted by C₁₋₅ alkyl, C₃₋₆ cycloalkyl, or C₆₋₁₂ aryl and the backbone comprises at least one rigidifying moiety connected to at least two atoms of the backbone. The rigidifying moiety is a double bond, triple bond, saturated or unsaturated C₃₋₉ cycloalkyl, or C₆₋₁₂ aryl.

In an embodiment, group X is:

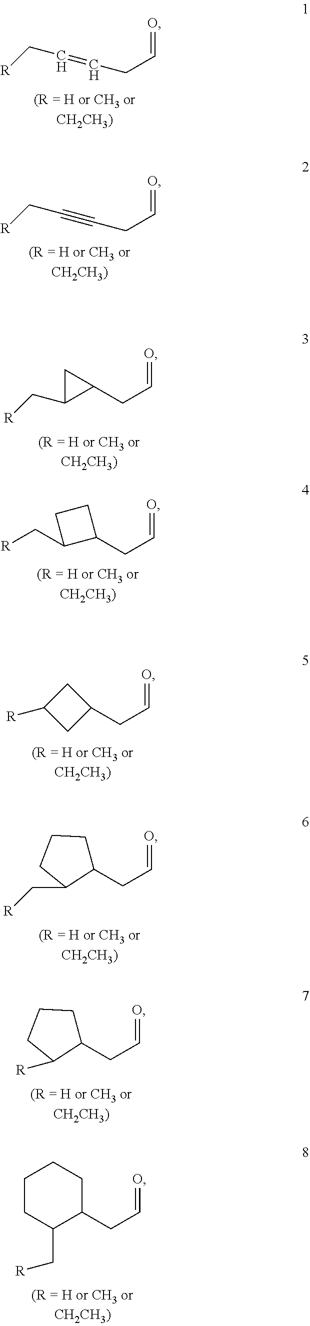

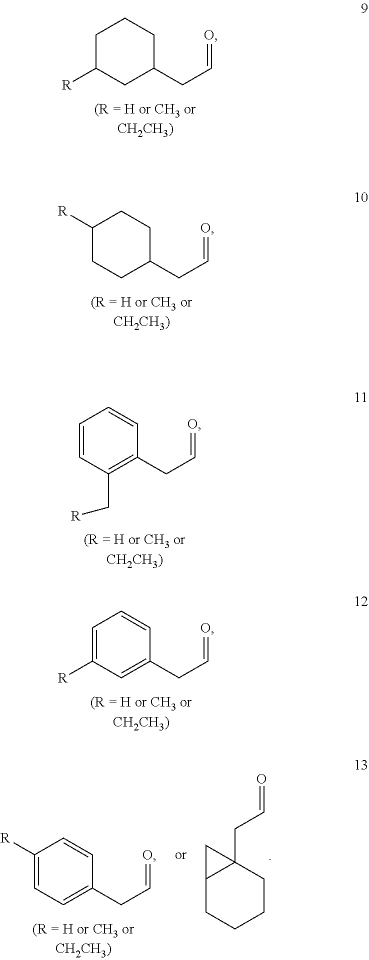

In an embodiment, in formula B, A30-A44 are: (a) absent; (b) an amino acid sequence corresponding to positions 30-44 of a native GHRH peptide (SEQ ID NO: 3), or (c) the amino acid sequence of (b) having a 1-14 amino acid deletion from its C-terminus.

In an embodiment, the GHRH peptide is a polypeptide comprising the amino acid sequence of SEQ ID NO: 4.

In an embodiment, the GHRH molecule is (hexenoyl trans-3)hGHRH₍₁₋₄₄₎NH₂ (SEQ ID NO: 1) or a pharmaceutically acceptable salt thereof. trans-3-hexenoyl]hGHRH₍₁₋₄₄₎ amide (also referred to as tesamorelin and (hexenoyl trans-3)hGHRH(1-44)NH₂) is a synthetic human GHRH (hGHRH) analog that comprises the 44-amino acid sequence of hGHRH on which a hexenoyl moiety, a C₆ side chain, has been anchored on the amino-terminal tyrosine residue. The structure of [trans-3-hexenoyl]hGHRH₍₁₋₄₄₎ amide is depicted at FIG. 8.

The term “pharmaceutically acceptable salt” refers to a salt of a GHRH molecule (e.g., trans-3-hexenoyl-GHRH₍₁₋₄₄₎-NH₂) that is pharmacologically acceptable and substantially non-toxic to the subject to which it is administered. More specifically, these salts retain the biological effectiveness and properties of the GHRH molecules (e.g., trans-3-hexenoyl-GHRH₍₁₋₄₄₎-NH₂) and are formed from suitable non-toxic organic or inorganic acids or bases.

For example, these salts include acid addition salts of GHRH molecules (e.g., trans-3-hexenoyl-GHRH₍₁₋₄₄₎-NH₂) which are sufficiently basic to form such salts. Such acid addition salts include acetates, adipates, alginates, lower alkanesulfonates such as a methanesulfonates, trifluoromethanesulfonatse or ethanesulfonates, arylsulfonates such as a benzenesulfonates, 2-naphthalenesulfonates, or toluenesulfonates (also known as tosylates), ascorbates, aspartates, benzoates, benzenesulfonates, bisulfates, borates, butyrates, citrates, camphorates, camphorsulfonates, cinnamates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydrogen sulphates, 2-hydroxyethanesulfonates, itaconates, lactates, maleates, mandelates, methanesulfonates, nicotinates, nitrates, oxalates, pamoates, pectinates, perchlorates, persulfates, 3-phenylpropionates, phosphates, picrates, pivalates, propionates, salicylates, succinates, sulfates, sulfonates, tartrates, thiocyanates, undecanoates and the like.

Additionally, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website).

Such salts can be formed quite readily by those skilled in the art using standard techniques. Indeed, the chemical modification of a pharmaceutical compound (i.e., drug) into a salt is a technique well known to pharmaceutical chemists, (See, e.g., H. Ansel et. al., Pharmaceutical Dosage Forms and Drug Delivery Systems (6^{th} Ed. 1995) at pp. 196 and 1456-1457). Salts of the trans-3-hexenoyl-GHRH₍₁₋₄₄₎-NH₂ may be formed, for example, by reacting the trans-3-hexenoyl-GHRH₍₁₋₄₄₎-NH₂ with an amount of acid or base, such as an equivalent amount, in a medium such as one in which the salt precipitates or in an aqueous medium followed by lyophilization.

In an embodiment, the pharmaceutically acceptable salt of the GHRH molecule, preferably trans-3-hexenoyl-GHRH₍₁₋₄₄₎-NH₂, is an acetate salt.

In an embodiment, the GHRH molecule, preferably trans-3-hexenoyl-GHRH₍₁₋₄₄₎-NH₂, or pharmaceutically acceptable salt thereof, is present in a pharmaceutical composition at a dose of about 1 mg/ml to about 10 mg/ml. In a further embodiment, the GHRH molecule, preferably trans-3-hexenoyl-GHRH₍₁₋₄₄₎-NH₂, or pharmaceutically acceptable salt thereof is present in a pharmaceutical composition at a dose of about 1 mg/ml to about 10 mg/ml, preferably about 1 mg/ml to about 8 mg/ml or about 4 mg/ml to about 8 mg/ml, for example about 1 mg/ml, about 2 mg/ml, about 3 mg/ml, about 4 mg/ml, about 5 mg/ml, about 6 mg/ml, about 7 mg/ml, or about 8 mg/ml.

In an embodiment, the GHRH molecule, preferably trans-3-hexenoyl-GHRH₍₁₋₄₄₎-NH₂, or pharmaceutically acceptable salt thereof is present in a pharmaceutical composition comprising one or more pharmaceutically acceptable excipients.

The term “pharmaceutically acceptable excipient” as used herein has its normal meaning in the art and is any ingredient that is not an active ingredient (drug) itself. Excipients include for example binders, lubricants, diluents, bulking agents (fillers), thickening agents, disintegrants, plasticizers, coatings, barrier layer formulations, lubricants, stabilizing agent, release-delaying agents and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present composition is not limited in these respects. In certain embodiments, the pharmaceutical composition comprises one or more excipients, including for example and without limitation, one or more binders (binding agents), thickening agents, surfactants, diluents, release-delaying agents, colorants, flavoring agents, fillers, disintegrants/dissolution promoting agents, lubricants, plasticizers, silica flow conditioners, glidants, anti-caking agents, anti-tacking agents, stabilizing agents, anti-static agents, swelling agents and any combinations thereof. As those of skill would recognize, a single excipient can fulfill more than two functions at once, e.g., can act as both a binding agent and a thickening agent. As those of skill will also recognize, these terms are not necessarily mutually exclusive. Therapeutic formulations are prepared using standard methods known in the art by mixing the active ingredient having the desired degree of purity with one or more optional pharmaceutically acceptable carriers, excipients and/or stabilizers. The excipient(s) may be suitable, for example, for intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration (see Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7^{th} edition, Pharmaceutical Press). In an embodiment, the pharmaceutical composition is an injectable composition. In an embodiment, the pharmaceutical composition comprises one or more excipients for subcutaneous administration/injection.

Methods to measure the amount/level of proteins in a biological sample are well known in the art. Protein levels may be detected directly using a ligand binding specifically to the protein (mature protein), such as an antibody or a fragment thereof. In embodiments, such a binding molecule or reagent (e.g., antibody) is labeled/conjugated, e.g., radio-labeled, chromophore-labeled, fluorophore-labeled, or enzyme-labeled to facilitate detection and quantification of the complex (direct detection). Alternatively, protein levels may be detected indirectly, using a binding molecule or reagent, followed by the detection of the [protein/binding molecule or reagent] complex using a second ligand (or second binding molecule) specifically recognizing the binding molecule or reagent (indirect detection). Such a second ligand may be radio-labeled, chromophore-labeled, fluorophore-labeled, or enzyme-labeled to facilitate detection and quantification of the complex. Enzymes used for labeling antibodies for immunoassays are known in the art, and the most widely used are horseradish peroxidase (HRP) and alkaline phosphatase (AP). Examples of binding molecules or reagents include antibodies (monoclonal or polyclonal), natural or synthetic ligands, and the like.

Examples of methods to measure the amount/level of protein in a sample include, but are not limited to: Western blot, immunoblot, enzyme-linked immunosorbent assay (ELISA), “sandwich” immunoassays, radioimmunoassay (RIA), Proximity Extension Assay (PEA), immunoprecipitation, surface plasmon resonance (SPR), chemiluminescence, fluorescent polarization, phosphorescence, immunohistochemical (IHC) analysis, matrix-assisted laser desorption/ionization time-of-flight (MALDI-TOF) mass spectrometry, microcytometry, microarray, antibody array, microscopy (e.g., electron microscopy), flow cytometry, proteomic-based assays, and assays based on a property or activity of the protein including but not limited to ligand binding or interaction with other protein partners, enzymatic activity, fluorescence. For example, if the protein of interest is a kinase known to phosphorylate a given target, the level or activity of the protein of interest may be determined by measuring the level of phosphorylation of the target in the presence of the test compound. If the protein of interest is a transcription factor known to induce the expression of one or more given target gene(s), the level or activity of the protein of interest may be determined by the measuring the level of expression of the target gene(s). In an embodiment, the amount/level of VEGFA, TGFB1, and/or CSF1 in the sample is measured by Proximity Extension Assay (PEA). PEA is an affinity-based assay that characterizes abundance levels of pre-determined sets of proteins. Each protein is targeted by a unique pair of oligonucleotide-labeled antibodies. When in close proximity, the oligonucleotides undergo a proximity-dependent DNA polymerization event to form a PCR target sequence. The resultant DNA sequence is detected and quantified using standard real-time PCR. PEA gives protein abundance levels of Normalized Protein eXpression (NPX) on a log₂-scale.

In an embodiment, the above-noted measuring protein levels of VEGFA, TGFB1, or CSF1 comprises contacting the biological sample with a ligand that specifically binds to the protein(s), such as an antibody or antigen-binding fragment thereof that specifically binds to VEGFA, TGFB1, or CSF1, and measuring the amount of complexes between VEGFA, TGFB1, or CSF1 and the ligand (e.g., antibody or antigen-binding fragment thereof). The term “antibody or antigen-binding fragment thereof” as used herein refers to any type of antibody/antibody fragment including monoclonal antibodies (including full-length monoclonal antibodies), polyclonal antibodies, multispecific antibodies, humanized antibodies, CDR-grafted antibodies, chimeric antibodies and antibody fragments so long as they exhibit the desired antigenic specificity/binding activity. Antibody fragments comprise a portion of a full-length antibody, generally an antigen binding or variable region thereof. Examples of antibody fragments include Fab, Fab′, F(ab′)₂, and Fv fragments, diabodies, linear antibodies, single-chain antibody molecules, single domain antibodies (e.g., from camelids), shark NAR single domain antibodies, and multispecific antibodies formed from antibody fragments. Antibody fragments can also refer to binding moieties comprising CDRs or antigen binding domains including, but not limited to, V_{H} regions (V_{H}, V_{H}-V_{H}), anticalins, PepBodies, antibody-T-cell epitope fusions (Troybodies) or Peptibodies.

In an embodiment, the antibody or antigen-binding fragment thereof is labelled. The antibody or antigen-binding fragment thereof may be labeled with one or more labels such as a biotin label, a fluorescent label, an enzyme label, a coenzyme label, a chemiluminescent label, or a radioactive isotope label. In an embodiment, the antibody or antigen-binding fragment thereof is labelled with a detectable label/moiety, for example a fluorescent moiety (fluorophore). Useful detectable labels include fluorescent compounds (e.g., fluorescein isothiocyanate, Texas red, rhodamine, fluorescein, Alexa Fluor® dyes, and the like), radiolabels, enzymes (e.g., horseradish peroxidase, alkaline phosphatase and others commonly used in an protein detection assays), streptavidin/biotin, and colorimetric labels such as colloidal gold, colored glass or plastic beads (e.g., polystyrene, polypropylene, latex, etc.). Chemiluminescent compounds may also be used. In another embodiment, the antibody or antigen-binding fragment thereof is conjugated to an oligonucleotide, e.g., to perform Proximity Extension Assay, as described above.

In an embodiment, the ligand that specifically binds to the protein(s) (e.g., an antibody or antigen-binding fragment thereof that specifically binds to VEGFA, TGFB1, or CSF1), is attached or immobilized on a solid support. The solid support may be any solid support which permits the binding (e.g., immobilization) of the ligand and which may be used for the desired application. It includes for example glass or plastic plates/slides. In an embodiment, the above-mentioned solid support is a plastic plate/slide. In embodiments, the above-mentioned plates/slides may be modified (e.g., coated, chemically modified, derivatized) prior to immobilization of the ligand. In an embodiment, the solid support is modified to permit or facilitate the covalent or non-covalent immobilization of the ligand, using any method known in the art. The solid support may be either amino- or carboxy-functionalized, depending on whether immobilization of the ligand through its C- or N-terminal end is desired. The solid support may be modified/coated using any conventional moiety capable of binding to a corresponding moiety (affinity tag) conjugated to the ligand, e.g., using typical affinity tags-based systems such as NTA—“His-Tag” systems, biotin—avidin/streptavidin systems, glutathione S-transferase (GST)—glutathione systems, Maltose Binding Protein (MBP)—amylose systems, as well as antigen—antibody systems.

In an embodiment, the above-mentioned method comprises a step of normalizing the protein levels, i.e., normalization of the measured levels of the above-noted proteins against a stably expressed control protein (or housekeeping protein) to facilitate the comparison between different samples. “Normalizing” or “normalization” as used herein refers to the correction of raw protein level values/data between different samples for sample to sample variations, to take into account differences in “extrinsic” parameters such as protein quality, efficiency of purification, etc., i.e., differences not due to actual “intrinsic” variations in proteins in the samples. Such normalization is performed by correcting the raw protein level values/data for a test protein (or protein of interest, i.e., VEGFA, TGFB1, and/or CSF1) based on the protein level values/data measured for one or more “housekeeping” or “control” protein, i.e., whose levels are known to be constant (i.e., to show relatively low variability) in the biological sample under different experimental conditions. Thus, in an embodiment, the above-mentioned method further comprises measuring the level of expression of a housekeeping protein in the biological sample.

The raw levels of VEGFA, TGFB1, and/or CSF1 measured in the sample may be subjected to mathematical transformations prior to analysis, such as log transformations. In an embodiment, the methods described herein comprises performing a Log2 transformation of the raw levels of VEGFA, TGFB1, and/or CSF1 measured in the sample prior to analysis.

In accordance with the present disclosure, a biological sample (e.g., a medical/clinical sample) encompasses any sample (crude or processed) obtained from a subject/patient suspected of containing the one or more target proteins described herein (VEGFA, TGFB1, and CSF1). Such substance may originate from a variety of sources. In an embodiment, a sample suspected to contain one or more target proteins may be obtained from any tissue/organ and/or from bodily excretions or fluids. The sample, if need be, may be prepared using techniques known to a person skilled in the art including, without limitation, mechanical lysis, detergent extraction, sonication, electroporation, denaturants, etc., and may also be purified if need be. In further embodiments, the sample may be processed to obtain an extract thereof enriched in proteins, ranging from relatively crude to relatively pure protein preparations.

In an embodiment, the above-mentioned biological sample is a biological fluid, e.g., urine, saliva, lymph, or a blood-derived sample. The term “blood-derived sample” as used herein refers to blood (e.g., fresh blood, stored blood) or to a fraction thereof, such as serum, plasma and the like. It also refers to any sample that may be obtained following one or more purification, enrichment, and/or treatment steps using blood (obtained by venous puncture, for example) as starting material. In an embodiment, the biological sample is a blood-derived sample, in a further embodiment plasma.

The sample may be obtained from a subject who is suspected of suffering from NAFLD, for example a subject who has one or more symptoms of fatty liver and/or liver fibrosis. The subject may be suspected of suffering from NAFLD, or having been diagnosed for NAFLD, based on results of laboratory testing such as elevated liver enzymes alanine aminotransferase (ALT) and/or aspartate aminotransferase (AST), evidence of liver fat detected by imaging techniques, and/or liver biopsy. The term NAFLD refers to a chronic liver disease defined as the pathological presence of hepatic steatosis (>5% of the cross-sectional area of the liver occupied by fat vacuoles) in the absence of any secondary cause for hepatic fat accumulation, such as alcohol use, steatogenic medication, and hereditary disorders. NAFLD comprises a spectrum of disease that can be simplified into two categories: (1) Simple Steatosis (SS) or nonalcoholic fatty liver (NAFL), 70%-75% of cases, defined by excess liver fat without inflammation or cellular injury; and (2) nonalcoholic steatohepatitis (NASH), 25%-30% of cases, defined by the presence of excess liver fat with inflammation and cellular injury with or without perisinusoidal fibrosis. In an embodiment, the biological sample is from a subject suffering from or suspected of suffering from NAFL. In another embodiment, the biological sample is from a subject suffering from or suspected of suffering from NASH. In another embodiment, the subject is an HIV-infected subject, i.e., the subject suffers from HIV-associated NAFLD.

In an embodiment, the methods described herein further comprise performing one or more additional assays to assess/diagnose NAFLD/NASH in the subject. Such assays include for example determining the levels of liver enzymes such as alanine aminotransferase (ALT) and/or aspartate aminotransferase (AST) in a biological sample from the subject, performing an imaging of the liver using imaging techniques such as ultrasound, computerized tomography (CT) scans, magnetic resonance imaging (MRI), ultrasound elastography (USE), quantitative ultrasound-based techniques, magnetic resonance elastography (MRE), and magnetic resonance-based fat quantitation technique, or histological analysis of a liver sample (e.g., liver biopsy). Such additional assay(s) may be performed on patients suspected of suffering from NAFLD based on higher/increased levels of VEGFA, TGFB1, and/or CSF1 in their biological samples (relative to reference levels), as described herein.

In another aspect, the present disclosure provides an assay mixture for (a) assessing the severity of NAFLD in a patient over time, and/or (b) assessing the likelihood that a subject suffers from NAFLD, the assay mixture comprising: (i) a biological sample from a subject suffering from or suspected of suffering from NAFLD; and (ii) one or more reagents for determining/measuring the protein levels of VEGFA, TGFB1, and/or CSF1 in the sample. In an embodiment, the biological sample is a blood-derived sample, in a further embodiment plasma. In an embodiment, the biological sample is from a subject suffering from NAFLD. In another embodiment, the biological sample is from an HIV-infected subject.

In another aspect, the present disclosure provides a system for (a) assessing the severity of NAFLD in a patient over time, and/or (b) assessing the likelihood that a subject suffers from NAFLD, the system comprising: (i) a biological sample from a subject suffering from or suspected of suffering from NAFLD; and (ii) and one or more assays for determining/measuring the protein levels of VEGFA, TGFB1, and/or CSF1 in the sample. In an embodiment, the biological sample is a blood-derived sample, in a further embodiment plasma. In an embodiment, the biological sample is from a subject suffering from NAFLD. In another embodiment, the biological sample is from an HIV-infected subject.

In another aspect, the present disclosure provides a system for (a) assessing the severity of NAFLD in a patient over time, and/or (b) assessing the likelihood that a subject suffers from NAFLD, the system comprising: a sample analyzer configured to produce a signal corresponding to the protein levels of VEGFA, TGFB1, and/or CSF1 in a biological sample of the subject; and a computer sub-system programmed to calculate, based on the one or more of the protein levels, whether the signal is higher or lower than a reference value. In various embodiments, the system further comprises the biological sample. In an embodiment, the biological sample is a blood-derived sample, in a further embodiment plasma. In an embodiment, the biological sample is from a subject suffering from NAFLD. In another embodiment, the biological sample is from an HIV-infected subject.

In another aspect, the present disclosure relates to a kit for use in (a) assessing the severity of NAFLD in a patient over time, and/or (b) assessing the likelihood that a subject suffers from NAFLD, the kit comprising reagents for measuring protein levels of VEGFA, TGFB1, and/or CSF1 in a biological sample; and instructions for correlating the protein levels of VEGFA, TGFB1, and/or CSF1 with the severity of NAFLD and/or the likelihood of suffering from NAFLD.

In an embodiment, the reagents in the assay mixture, system and/or kit comprise, for example, ligands for VEGFA, TGFB1, and/or CSF1 (e.g., antibody(ies) or fragments thereof), solution(s), buffer(s), nucleic acid amplification reagent(s) (e.g., DNA polymerase, DNA polymerase cofactor, dNTPs), nucleic acid hybridization/detection reagent(s), and/or reagents for detecting antigen-antibody complexes, etc. In an embodiment, the reagents comprise ligands (e.g., antibody(ies) or fragments thereof) for at least two of VEGFA, TGFB1, and/or CSF1. In an embodiment, the reagents comprise ligands (e.g., antibody(ies) or fragments thereof) for (i) VEGFA and TGFB1; (ii) VEGFA and CSF1; (iii) TGFB1 and CSF1; or (iv) VEGFA, TGFB1 and CSF1. In an embodiment, the assay mixture, system and/or kit comprise an array comprising ligands (e.g., antibody(ies) or fragments thereof) for (i) VEGFA and TGFB1; (ii) VEGFA and CSF1; (iii) TGFB1 and CSF1; or (iv) VEGFA, TGFB1 and CSF1.

In an embodiment, the kit according to the present disclosure may be divided into separate packages or compartments containing the respective reagent components explained above.

In addition, such a kit may optionally comprise one or more of the following: (1) instructions for using the reagents for performing the methods described herein and/or for interpreting the results obtained; (2) one or more containers; and/or (3) appropriate controls/standards. Such a kit can include reagents for collecting a biological sample from a patient and reagents for processing the biological sample.

Informational material included in the kits can be descriptive, instructional, marketing or other material that relates to the methods described herein and/or the use of the reagents for the methods described herein. For example, the informational material of the kit can contain contact information, e.g., a physical address, email address, website, or telephone number, where a user of the kit can obtain substantive information about performing the method described herein and interpreting the results.

The kits featured herein can also provide software necessary to infer the severity of NAFLD in a patient and/or the likelihood that a subject suffers from NAFLD from the protein level data.

In another aspect, there is provided the use of the kit or assay mixture described herein for (a) assessing the severity of NAFLD in a patient over time, and/or (b) assessing the likelihood that a subject suffers from NAFLD.

### MODE(S) FOR CARRYING OUT THE INVENTION

The present disclosure is illustrated in further details by the following non-limiting examples.

### EXAMPLE 1: MATERIALS AND METHODS

### Study Design

A randomized, double blind trial in which individuals with HIV-associated NAFLD were assigned to receive the growth hormone-releasing hormone (GHRH) analogue tesamorelin 2 mg daily or identical placebo for 12 months (7). Leveraging plasma specimens from this trial, the current study builds significantly on prior, purely transcriptomic analyses (8) to examine specific proteins. Changes in circulating levels of proteins corresponding to top leading-edge genes in pathways responsive to tesamorelin between treatment groups were investigated, and the relationships of these proteins to histologic, radiographic, and transcriptomic indices was assessed to identify plasmatic markers of NAFLD/NASH and elucidate potential mechanisms of tesamorelin response.

61 men and women 18-70 years old who had documented HIV infection and liver steatosis as defined by hepatic fat fraction ≥5% on ¹H-magnetic resonance spectroscopy (¹H-MRS) were enrolled. Participants were required to have been on stable antiretroviral therapy (ART) for ≥3 months with CD4⁺ T cell count>100 cells/mm³ and HIV viral load<400 copies/mL. Exclusion criteria included excess alcohol use (>20 g daily for women or >30 g daily for men), active hepatitis B or C, other known hepatic disease, cirrhosis, and inadequately controlled diabetes mellitus (HbA1c≥7%). Participants were enrolled at the Massachusetts General Hospital (MGH, Boston, MA) and the National Institutes of Health (NIH, Bethesda, MD) between Aug. 20, 2015 and Jan. 16, 2019. Informed consent in writing was obtained from each participant. All methods were carried out in accordance with guidelines and regulations.

### Study Procedures

Study procedures for the parent clinical trial have been described in detail elsewhere (7, 9). All study procedures were conducted in a fasting state. In brief, hepatic ¹H-MRS was performed for measurement of hepatic fat fraction at baseline and 12 months. An ultrasound-guided percutaneous liver biopsy yielding two cores also was completed at each time point. The first core was fixed in formalin, and subsequently underwent histopathologic review by a single expert pathologist blinded to treatment (D.E.K., National Institutes of Health). Histological scoring, including NAFLD Activity Score (NAS) and fibrosis stage, was performed according to the Nonalcoholic Steatohepatitis Clinical Research Network scoring system (10). The second core was placed in an RNA stabilization reagent (RNAlater®, Qiagen) and stored at −80° C. for gene expression analyses. Blood specimens were collected at baseline and 12 months and stored at −80° C. Serum IGF-1 was measured using standard techniques (Quest Laboratories).

### Hepatic Transcriptomic Assessment

Liver tissue underwent RNA extraction, cDNA library construction, and Illumina sequencing using methods that have been previously described (9). To identify pathways differentially modulated from pre- to post-treatment time points between tesamorelin- and placebo-treated participants, GSEA was performed using the desktop module from the Broad Institute (www.broadinstitute.org/gsea/). Gene sets used included the Molecular Signatures Database (MsigDB) hallmark gene set collection (11) and custom gene sets pertaining to HCC prognosis (9). GSEA leading-edge genes were the subset of genes in a significantly enriched gene set that accounted for the enrichment signal and were used for the subsequent quantification of pathway gene expression. Gene sets with false discovery rate (FDR) <0.05 were considered enriched.

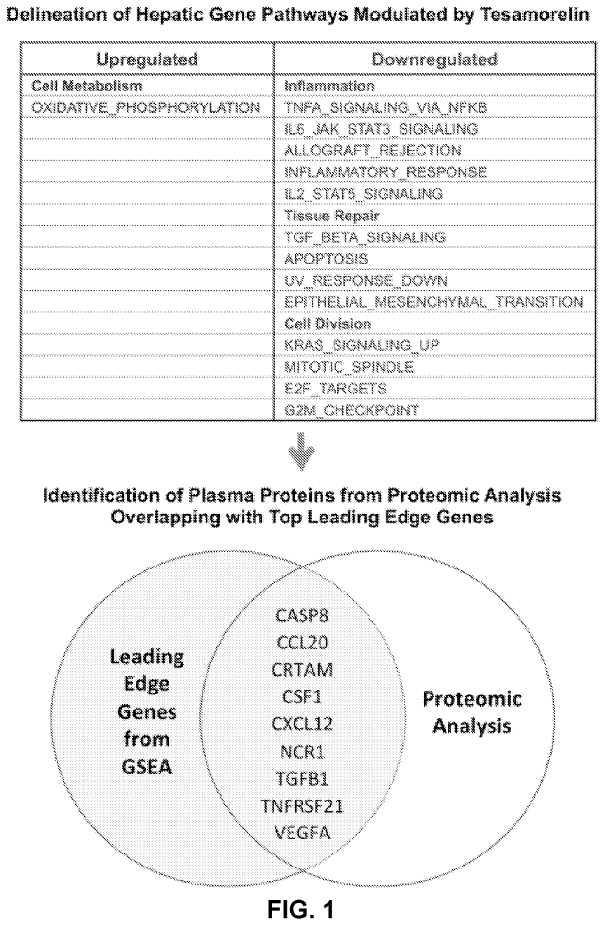

Utilizing this approach, 14 hallmark gene pathways that were differentially regulated by tesamorelin versus placebo were previously discovered. In this regard, a gene set pertaining to oxidative phosphorylation was upregulated with treatment. Furthermore, 13 gene sets involved in inflammation, tissue repair, and cell division were downregulated among tesamorelin-treated individuals (FIG. 1). The RNA-Seq data were submitted to the Gene Expression Omnibus repository at the National Center for Biotechnology Information (accession number GSE150026).

### Plasma Proteomic Assessment

For this analysis, change in targeted proteins over 12 months was assessed using an Olink® Multiplex proximity extension assay (PEA) platform. The PEA is an affinity-based assay that characterizes abundance levels of pre-determined sets of proteins. Each protein is targeted by a unique pair of oligonucleotide-labeled antibodies. When in close proximity, the oligonucleotides undergo a proximity-dependent DNA polymerization event to form a PCR target sequence. The resultant DNA sequence is detected and quantified using standard real-time PCR on the Fluidigm BioMark™ HD real-time PCR platform. The PEA gives protein abundance levels of Normalized Protein eXpression (NPX) on a log₂-scale. Assay characteristics including detection limits and measurements of assay performance are available from the manufacturer (Olink, Uppsala, Sweden). Specificity is high due to the precision of the methodology, which enabled assessment of change over time. Across all proteins within the high-multiplex panel utilized (below), the mean intra-assay and inter-assay variation were reported as 8.3% and 11.5%, respectively.

### Targeted Proteomic Analysis

An objective of the current study was to delineate potential response pathways of tesamorelin effects in NAFLD, and to determine a protein signature that might be used to detect a treatment response to tesamorelin among patients with NAFLD. To do so, all plasma proteins within a high-multiplex panel of nearly 100 proteins (Olink Immuno-Oncology; see www. olink. com for the complete protein list) that were found to overlap with top leading genes from tesamorelin-responsive gene sets were flagged (8). Among this targeted set of proteins, changes in plasma levels by treatment status were compared. Proteins found to be differentially modulated by tesamorelin relative to placebo were then examined in relation to radiographic, histologic, and transcriptomic indices of NAFLD severity both at baseline and longitudinally. As a surrogate for fibrosis stage, a gene-level fibrosis score derived from the hepatic expression of 18 genes shown to correlate with fibrosis (11) was utilized, which was validated in the current sample to histological changes as previously described (8). Changes in levels of these proteins were also related to changes in their corresponding hepatic transcript level and change in serum IGF-1.

Continuous variables were expressed as mean ±standard deviation, whereas categorical variables were indicated as a frequency (%). Differences between groups were compared using a two-tailed independent samples t-test for continuous variables and chi-square test for categorical variables. Correlations were assessed with Pearson correlation coefficient. A value of P≤0.05 was the pre-defined threshold for statistical significance. Statistical analyses were performed using JMP Pro 14 (SAS Institute Inc., Cary, North Carolina, USA).

TABLE 1
Overlap of Proteins Studied with Top Leading-edge
Genes Within Differentially Modulated Gene Sets

| Plasma protein | Gene set with corresponding top leading edge gene |
|---|---|
| CASP8 | APOPTOSIS |
| CCL20 | INFLAMMATORY_RESPONSE, RAS_SIGNALING_UP, |
| | TNFA_SIGNALING_VIA_NFKB |
| CRTAM | ALLOGRAFT_REJECTION |
| CSF1 | IL6_JAK_STAT3_SIGNALING, INFLAMMATORY_RESPONSE |
| CXCL12 | EPITHELIAL_MESENCHYMAL_TRANSITION |
| NCR1 | ALLOGRAFT_REJECTION |
| TGFB1 | TGF_BETA_SIGNALING, IL6_JAK_STAT3_SIGNALING |
| TNFRSF21 | TNFRSF21 |
| VEGFA | TNFA_SIGNALING_VIA_NFKB |

Abbreviations: CASP8, caspase 8; CCL20, C-C motif chemokine ligand 20; CRTAM, cytotoxic and regulatory T-cell molecule; CSF1, macrophage colony stimulating factor 1; CXCL12, C-X-C motif chemokine ligand 12; NCR1, natural cytotoxicity triggering receptor 1; TGFB1, transforming growth factor beta 1; TNFRSF21, tumor necrosis factor receptor superfamily member 21; VEGFA, vascular endothelial growth factor A.

### EXAMPLE 2: CHARACTERISTICS OF STUDY PARTICIPANTS

Of 61 participants with HIV-associated NAFLD in the randomized-controlled trial, 58 individuals had a plasma protein panel obtained at baseline that was available for analysis. Moreover, 44 of these individuals (20 assigned to tesamorelin, 24 assigned to placebo) had plasma protein panels repeated at 12 months. Characteristics of each treatment group in the overall sample are summarized in Table 2 and have been described previously (7). Tesamorelin and placebo groups were well balanced with respect to key clinical variables. Briefly, participants (53±7 years old, 79% male) had well-controlled HIV infection for 17±9 years. All subjects received stable ART with 64% on integrase inhibitor-based regimens. Baseline hepatic fat content was 14±8% as measured by hepatic ¹H-MRS. A total of 33% and 43% had histologic evidence of NASH and fibrosis, respectively, on initial liver biopsy.

TABLE 2
Baseline Demographic and Clinical Characteristics

| | Overall Sample | | Subset with Paired Protein Panels | |
|---|---|---|---|---|
| | Tesamorelin | Placebo | Tesamorelin | Placebo |
| | (n = 31) | (n = 30) | (n = 20) | (n = 24) |
| Age (years) | 52 ± 8 | 54 ± 7 | 53 ± 6 | 54 ± 7 |
| % Male | 77 | 80 | 70 | 79 |
| % Race | | | | |
| White | 68 | 63 | 55 | 71 |
| Black | 26 | 33 | 40 | 29 |
| Other | 6 | 3 | 5 | 0 |
| % Hispanic | 19 | 10 | 10 | 8 |
| Duration of HIV infection | 16 ± 9 | 18 ± 8 | 17 ± 10 | 18 ± 8 |
| (years) | | | | |
| CD4 count (cells/mm³) | 733 ± 290 | 798 ± 260 | 740 ± 265 | 792 ± 269 |
| log HIV viral load | 0.34 ± 0.59 | 0.50 ± 0.74 | 0.39 ± 0.61 | 0.41 ± 0.66 |
| % Current | | | | |
| Antiretroviral Use | | | | |
| NRTI | 87 | 97 | 85 | 96 |
| NNRTI | 39 | 37 | 30 | 38 |
| PI | 29 | 20 | 25 | 25 |
| Integrase Inhibitor | 68 | 60 | 80 | 58 |
| % Type 2 Diabetes | 13 | 13 | 5 | 13 |
| Liver biopsy length (mm) | 13 ± 3 | 13 ± 3 | 14 ± 2 | 13 ± 3 |
| Hepatic Fat Fraction (%) | 13 ± 8 | 15 ± 9 | 15 ± 9 | 15 ± 9 |
| % NASH | 34 | 31 | 22 | 35 |
| % Fibrosis | 48 | 38 | 39 | 39 |
| Stage 1 | 14 | 17 | 17 | 13 |
| Stage 2 | 21 | 14 | 17 | 17 |
| Stage 3 | 14 | 7 | 6 | 9 |

There were no statistically significant differences between groups at baseline for any of the variables shown above.
Continuous variables are presented as mean ± standard deviation.
Abbreviations: NASH, nonalcoholic steatohepatitis; NNRTI, non-nucleoside reverse transcriptase inhibitor; NRTI, nucleoside reverse transcriptase inhibitor; PI, protease inhibitor.

### EXAMPLE 3: PLASMA PROTEINS DIFFERENTIALLY REGULATED BY TESAMORELIN

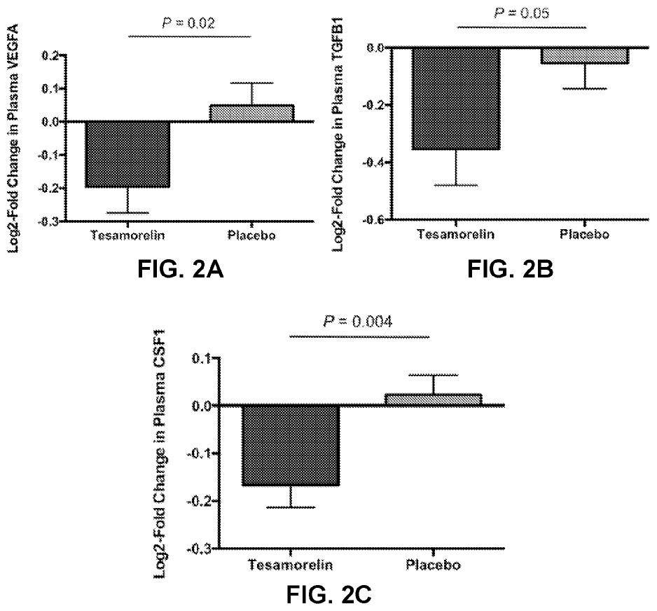

Nine plasma proteins were identified as corresponding to top leading edge genes modulated by tesamorelin (FIG. 1). These leading-edge genes were contained within gene sets pertaining to inflammation, tissue repair, and cell division (Table 1) that were downregulated by treatment with tesamorelin versus placebo. Of these proteins, treatment with tesamorelin led to reductions in plasma VEGFA (log₂-fold change −0.20±0.35 vs. 0.05±0.34, P=0.02), TGFB1 (log₂-fold change −0.35±0.56 vs. −0.05±0.43, P=0.05), and CSF1 (log₂-fold change −0.17±0.21 vs. 0.02±0.20, P=0.004) compared to placebo (FIGS. 2A-2C). Moreover, plasma CCL20 tended to decrease with tesamorelin though the difference between groups did not reach statistical significance (log₂-fold change −0.28±0.88 vs. 0.20±0.79, P=0.06). The effect of treatment versus placebo on all 9 plasma proteins is summarized in Table 3.

TABLE 3
Effects of Tesamorelin Versus Placebo on Select Plasma Proteins

| | Log2-Baseline | | Log2-Fold change | | |
|---|---|---|---|---|---|
| Plasma | Tesamorelin | Placebo | Tesamorelin | Placebo | |
| protein | (n = 20) | (n = 24) | (n = 20) | (n = 24) | P-value |
| CASP8 | 4.91 (4.52, | 4.76 (4.39, | −0.37 ± 0.72 | −0.02 ± 0.65 | 0.10 |
| | 5.30) | 5.13) | | | |
| CCL20 | 7.53 (6.85, | 7.15 (6.62, | −0.28 ± 0.88 | 0.20 ± 0.79 | 0.06 |
| | 8.21) | 7.68) | | | |
| CRTAM | 5.71 (5.40, | 6.08 (5.89, | −0.35 ± 0.73 | −0.04 ± 0.50 | 0.10 |
| | 6.02) | 6.27) | | | |
| CSF1 | 9.84 (9.77, | 9.79 (9.72, | −0.17 ± 0.21 | 0.02 ± 0.20 | 0.004 |
| | 9.92) | 9.85) | | | |
| CXCL12 | 1.16 (1.08, | 1.15 (1.10, | −0.03 ± 0.18 | −0.003 ± 0.17 | 0.54 |
| | 1.25) | 1.20) | | | |
| NCR1 | 3.05 (2.81, | 3.21 (2.99, | −0.20 ± 0.52 | −0.08 ± 0.31 | 0.37 |
| | 3.29) | 3.42) | | | |
| TGFB1 | 8.99 (8.73, | 8.82 (8.67, | −0.35 ± 0.56 | −0.05 ± 0.43 | 0.05 |
| | 9.24) | 8.98) | | | |
| TNFRSF21 | 7.57 (7.44, | 7.62 (7.52, | −0.13 ± 0.22 | −0.05 ± 0.23 | 0.25 |
| | 7.71) | 7.72) | | | |
| VEGFA | 8.13 (7.97, | 7.92 (7.81, | −0.20 ± 0.35 | 0.05 ± 0.34 | 0.02 |
| | 8.29) | 8.03) | | | |

Bold text denotes P < 0.05. Data are represented as mean (95% confidence interval) or mean ± SD.
Abbreviations: CASP8, caspase 8; CCL20, C-C motif chemokine ligand 20; CRTAM, cytotoxic and regulatory T-cell molecule; CSF1, macrophage colony stimulating factor 1; CXCL12, C-X-C motif chemokine ligand 12; NCR1, natural cytotoxicity triggering receptor 1; TGFB1, transforming growth factor beta 1; TNFRSF21, tumor necrosis factor receptor superfamily member 21; VEGFA, vascular endothelial growth factor A.

### EXAMPLE 4: ASSOCIATION OF KEY PLASMA PROTEINS WITH NAFLD PHENOTYPE

Given their differential regulation by tesamorelin, the relationships of VEGFA, TGFB1, and CSF1 with NAFLD phenotype (Table 4) was next studied. At baseline, in the overall sample, plasma CSF1 level directly correlated with NAS score (r=0.38, P=0.004) and gene-level fibrosis score (r=0.37, P=0.03). In contrast, VEGFA and TGFB1 were not found to be associated with either of these parameters. Furthermore, there was no baseline relationship of VEGFA, TGFB1, or CSF1 with hepatic fat fraction.

TABLE 4
Relationships of Plasma Proteins Downregulated
by Tesamorelin with NAFLD Severity
A) Baseline

| | | Hepatic Fat | Fibrosis-Related |
|---|---|---|---|
| Log₂ | NAS Score | Fraction (%) | Gene Score |
| Plasma Protein | (n = 58) | (n = 61) | (n = 37) |
| VEGFA | r = −0.10, P = 0.48 | r = −0.001, P = 0.99 | r = −0.09, P = 0.60 |
| TGFB1 | r = 0.31, P = 0.02 | r = 0.30, P = 0.02 | r = 0.22, P = 0.19 |
| CSF1 | r = 0.38, P = 0.004 | r = 0.21, P = 0.12 | r = 0.37, P = 0.03 |

B) Longitudinal

| Log₂-Fold | Change in NAS Score | |
|---|---|---|
| Change in | Tesamorelin | Placebo |
| Plasma Protein | (n = 18) | (n = 22) |
| VEGFA | r = 0.62, P = 0.006 | r = 0.11, P = 0.62 |
| TGFB1 | r = 0.31, P = 0.22 | r = 0.24, P = 0.29 |
| CSF1 | r = 0.50, P = 0.04 | r = 0.21, P = 0.35 |

| Log₂-Fold | Change in Hepatic Fat Fraction (%) | |
|---|---|---|
| Change in | Tesamorelin | Placebo |
| Plasma Protein | (n = 19) | (n = 23) |
| VEGFA | r = 0.30, P = 0.22 | r = 0.12, P = 0.57 |
| TGFB1 | r = 0.17, P = 0.49 | r = 0.34, P = 0.11 |
| CSF1 | r = 0.13, P = 0.60 | r = 0.16, P = 0.46 |

| Log₂-Fold | Change in Fibrosis-Related Gene Score | |
|---|---|---|
| Change in | Tesamorelin | Placebo |
| Plasma Protein | (n = 17) | (n = 19) |
| VEGFA | r = 0.30, P = 0.24 | r = −0.04, P = 0.86 |
| TGFB1 | r = 0.61, P = 0.009 | r = −0.07, P = 0.79 |
| CSF1 | r = 0.64, P = 0.006 | r = 0.29, P = 0.23 |

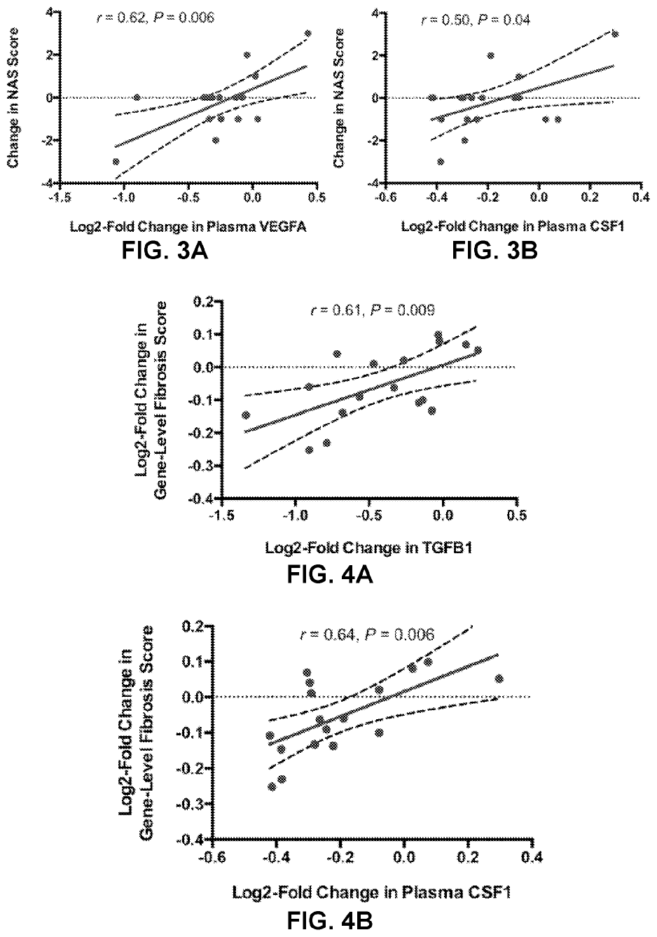

Within the tesamorelin-treated arm, reductions in plasma VEGFA (r=0.62, P=0.006) and CSF1 (r=0.50, P=0.04) strongly correlated with a decline in NAS score (FIGS. 3A, 3B and Table 4). Furthermore, among tesamorelin-treated participants, reductions in TGFB1 (r=0.61, P=0.009) and CSF1 (r=0.64, P=0.006) were associated with a decline in gene-level fibrosis score (FIGS. 4A, 4B). Changes in these 3 plasma proteins were not found to correlate with change in NAS score or gene-level fibrosis score among placebo-treated participants, or with change in hepatic fat fraction within either treatment group.

### EXAMPLE 5: ASSOCIATION OF CHANGES IN KEY PLASMA PROTEINS WITH CHANGES IN HEPATIC TRANSCRIPT LEVELS AND SERUM IGF-1

To elucidate the regulation of plasma VEGFA, TGFB1, and CSF1, their relationships with corresponding hepatic transcript levels and serum IGF-1 levels within the overall sample were next investigated. CSF1 exhibited a correlation between changes in plasma protein and hepatic transcript levels (r=0.50, P=0.002). Additionally, an increase in serum IGF-1 was associated with a linear decline in CSF1 (r=−0.38, P=0.01).

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. Maurice J B, Patel A, Scott A J, Patel K, Thursz M, Lemoine M. Prevalence and risk factors of nonalcoholic fatty liver disease in HIV-monoinfection. AIDS 2017;31:1621-1632.
2. Koethe J R, Jenkins C A, Lau B, Shepherd B E, Justice A C, Tate J P, Buchacz K, et al. Rising Obesity Prevalence and Weight Gain Among Adults Starting Antiretroviral Therapy in the United States and Canada. AIDS Res Hum Retroviruses 2016;32:50-58.
3. Grant P M, Kitch D, McComsey G A, Collier A C, Bartali B, Koletar S L, Erlandson K M, et al. Long-term body composition changes in antiretroviral-treated HIV-infected individuals. AIDS 2016;30:2805-2813.
4. Vodkin I, Valasek M, Bettencourt R, Cachay E, Loomba R. Clinical, biochemical and histological differences between HIV-associated NAFLD and primary NAFLD: a case-control study. Alimentary pharmacology & therapeutics 2015;41:368-378.
5. Fourman L T, Stanley T L, Zheng I, Pan C S, Feldpausch M N, Purdy J, Aepfelbacher J, et al. Clinical Predictors of Liver Fibrosis Presence and Progression in HIV-Associated NAFLD. Clin Infect Dis 2020.
6. Wong V W, Singal A K. Emerging medical therapies for non-alcoholic fatty liver disease and for alcoholic hepatitis. Transl Gastroenterol Hepatol 2019;4:53.
7. Stanley T L, Fourman L T, Feldpausch M N, Purdy J, Zheng I, Pan C S, Aepfelbacher J, et al. Effects of tesamorelin on non-alcoholic fatty liver disease in HIV: a randomised, double-blind, multicentre trial. Lancet HIV 2019;6:e821-e830.
8. Fourman L T, Billingsley J M, Agyapong G, Ho Sui S J, Feldpausch M N, Purdy J, Zheng I, et al. Effects of tesamorelin on hepatic transcriptomic signatures in HIV-associated NAFLD. JCI Insight 2020;5.
9. Kleiner D E, Brunt E M, Van Natta M, Behling C, Contos M J, Cummings O W, Ferrell L D, et al. Design and validation of a histological scoring system for nonalcoholic fatty liver disease. Hepatology 2005;41:1313-1321.
10. Liberzon A, Birger C, Thorvaldsdottir H, Ghandi M, Mesirov J P, Tamayo P. The Molecular Signatures Database (MSigDB) hallmark gene set collection. Cell Syst 2015;1:417-425.
11. Hoang S A, Oseini A, Feaver R E, Cole B K, Asgharpour A, Vincent R, Siddiqui M, et al. Gene Expression Predicts Histological Severity and Reveals Distinct Molecular Profiles of Nonalcoholic Fatty Liver Disease. Sci Rep 2019;9:12541.
12. Kitade M, Yoshiji H, Kojima H, Ikenaka Y, Noguchi R, Kaji K, Yoshii J, et al. Neovascularization and oxidative stress in the progression of non-alcoholic steatohepatitis. Mol Med Rep 2008;1:543-548.
13. Coulon S, Francque S, Colle I, Verrijken A, Blomme B, Heindryckx F, De Munter S, et al. Evaluation of inflammatory and angiogenic factors in patients with non-alcoholic fatty liver disease. Cytokine 2012;59:442-449.
14. Cayon A, Crespo J, Guerra A R, Pons-Romero F. [Gene expression in obese patients with non-alcoholic steatohepatitis]. Rev Esp Enferm Dig 2008;100:212-218.
15. Coulon S, Legry V, Heindryckx F, Van Steenkiste C, Casteleyn C, Olievier K, Libbrecht L, et al. Role of vascular endothelial growth factor in the pathophysiology of nonalcoholic steatohepatitis in two rodent models. Hepatology 2013;57:1793-1805.
16. Fabregat I, Moreno-Càceres J, Sanchez A, Dooley S, Dewidar B, Giannelli G, Ten Dijke P. TGF-β signalling and liver disease. FEBS J 2016;283:2219-2232.
17. Yang L, Roh Y S, Song J, Zhang B, Liu C, Loomba R, Seki E. Transforming growth factor beta signaling in hepatocytes participates in steatohepatitis through regulation of cell death and lipid metabolism in mice. Hepatology 2014;59:483-495.
18. Cayon A, Crespo J, Mayorga M, Guerra A, Pons-Romero F. Increased expression of Ob-Rb and its relationship with the overexpression of TGF-betal and the stage of fibrosis in patients with nonalcoholic steatohepatitis. Liver Int 2006;26:1065-1071.
19. Hamilton, J. A., Cook, A. D. & Tak, P. P. Anti-colony-stimulating factor therapies for inflammatory and autoimmune diseases. Nat. Rev. Drug Discov. 16, 53-70
20. Stutchfield B M, Antoine D J, Mackinnon A C, Gow D J, Bain C C, Hawley C A, Hughes M J, et al. CSF1 Restores Innate Immunity After Liver Injury in Mice and Serum Levels Indicate Outcomes of Patients With Acute Liver Failure. Gastroenterology 2015;149:1896-1909 e1814.
21. Naito M, Hayashi S, Yoshida H, Nishikawa S, Shultz L D, Takahashi K. Abnormal differentiation of tissue macrophage populations in ‘osteopetrosis’ (op) mice defective in the production of macrophage colony-stimulating factor. Am J Pathol 1991;139:657-667.
22. Radi Z A, Koza-Taylor P H, Bell R R, Obert L A, Runnels H A, Beebe J S, Lawton M P, et al. Increased serum enzyme levels associated with kupffer cell reduction with no signs of hepatic or skeletal muscle injury. Am J Pathol 2011;179:240-247.
23. Tosello-Trampont A C, Landes S G, Nguyen V, Novobrantseva T I, Hahn Y S. Kuppfer cells trigger nonalcoholic steatohepatitis development in diet-induced mouse model through tumor necrosis factor-a production. J Biol Chem 2012;287:40161-40172.
24. Li H, Zhou Y, Wang H, Zhang M, Qiu P, Zhang M, Zhang R, et al. Crosstalk Between Liver Macrophages and Surrounding Cells in Nonalcoholic Steatohepatitis. Front Immunol 2020;11:1169.
25. Rosenfeld M E, Ylä-Herttuala S, Lipton B A, Ord V A, Witztum J L, Steinberg D. Macrophage colony-stimulating factor mRNA and protein in atherosclerotic lesions of rabbits and humans. Am J Pathol 1992;140:291-300.
26. Sjaarda J, Gerstein H, Chong M, Yusuf S, Meyre D, Anand S S, Hess S, et al. Blood CSF1 and CXCL12 as Causal Mediators of Coronary Artery Disease. J Am Coll Cardiol 2018;72:300-310.

1. A method for assessing the severity of nonalcoholic fatty liver disease (NAFLD) in a patient over time, the method comprising
measuring a first protein level of Vascular Endothelial Growth Factor A (VEGFA), Transforming Growth Factor Beta 1 (TGFB1), and/or Colony Stimulating Factor 1 (CSF1) in a biological sample from the patient at a first time point;
measuring a second protein level of VEGFA, TGFB1, and/or CSF1 in a corresponding biological sample from the patient at a second, later time point;
wherein a decrease in the second protein level relative to the first protein level of VEGFA, TGFB1, and/or CSF1 between said first and second time points is indicative that NAFLD severity has regressed over time in the patient;
wherein an increase in the second protein level relative to the first protein level of VEGFA, TGFB1, and/or CSF1 between said first and second time points is indicative that NAFLD severity has progressed over time in the patient; and
wherein no change in the second protein level relative to the first protein level of VEGFA, TGFB1, and CSF1 between said first and second time points is indicative that NAFLD severity has been stable over time in the patient.

2-4. (canceled)

5. The method of claim 1, wherein NAFLD severity comprises the fibrosis score and/or the NAFLD Activity Score (NAS).

6. The method of claim 5, wherein NAFLD severity comprises the fibrosis score.

7. The method of claim 5, wherein NAFLD severity comprises the NAS.

8. The method of claim 1, wherein the patient has received a treatment against NAFLD between said first time point and said second time point.

9. The method of claim 8, wherein the treatment comprises administration of a Growth Hormone-Releasing Hormone (GHRH) molecule or an analog thereof.

10. The method of claim 9, wherein the treatment comprises administration of trans-3-hexenoyl-GHRH(1-44)-NH₂ or a pharmaceutically acceptable salt thereof.

11. The method of claim 1, wherein the biological sample is a plasma sample.

12. (canceled)

13. The method of claim 1, wherein measuring protein levels of VEGFA comprises contacting the biological sample with an antibody or antigen-binding fragment thereof that specifically binds to VEGFA, and measuring the amount of complexes between VEGFA and the antibody or antigen-binding fragment thereof; measuring protein levels of TGFB1 comprises contacting the biological sample with an antibody or antigen-binding fragment thereof that specifically binds to TGFB1, and measuring the amount of complexes between TGFB1 and the antibody or antigen-binding fragment thereof and/or measuring protein levels of CSF1 comprises contacting the biological sample with an antibody or antigen-binding fragment thereof that specifically binds to CSF1, and measuring the amount of complexes between CSF1 and the antibody or antigen-binding fragment thereof.

14-16. (canceled)

17. The method of claim 1, wherein said patient suffers from human immunodeficiency virus (HIV) infection.

18. A method for assessing the likelihood that a subject suffers from nonalcoholic fatty liver disease (NAFLD), the method comprising measuring protein levels of Vascular Endothelial Growth Factor A (VEGFA), Transforming Growth Factor Beta 1 (TGFB1), and/or Colony Stimulating Factor 1 (CSF1) in a biological sample from the subject, wherein a higher level of VEGFA, TGFB1, and/or CSF1 in the sample relative to a corresponding control level is indicative of an increased likelihood that the subject suffers from NAFLD.

19-21. (canceled)

22. The method of claim 18, wherein the NAFLD is nonalcoholic steatohepatitis (NASH).

23. The method of claim 18, wherein the NAFLD comprises liver fibrosis.

24. The method of claim 18, wherein the biological sample is a plasma blood derived sample.

25. (canceled)

26. The method of claim 18, wherein measuring protein levels of VEGFA comprises contacting the biological sample with an antibody or antigen-binding fragment thereof that specifically binds to VEGFA, and measuring the amount of complexes between VEGFA and the antibody or antigen-binding fragment thereof; measuring protein levels of TGFB1 comprises contacting the biological sample with an antibody or antigen-binding fragment thereof that specifically binds to TGFB1, and measuring the amount of complexes between TGFB1 and the antibody or antigen-binding fragment thereof and/or measuring protein levels of CSF1 comprises contacting the biological sample with an antibody or antigen-binding fragment thereof that specifically binds to CSF1, and measuring the amount of complexes between CSF1 and the antibody or antigen-binding fragment thereof.

27-31. (canceled)

32. The method of claim 18, wherein said subject suffers from human immunodeficiency virus (HIV) infection.

33. A method for treating nonalcoholic fatty liver disease (NAFLD), the method comprising identifying a subject having an increased likelihood of suffering from NAFLD using the method of claim 18, and administering a treatment against NAFLD to the subject.

34. The method of claim 33, wherein the treatment comprises administration of Growth Hormone-Releasing Hormone (GHRH) or an analog thereof.

35. The method of claim 34, wherein the treatment comprises administration of trans-3-hexenoyl-GHRH(1-44)-NH₂ or a pharmaceutically acceptable salt thereof.

36-37. (canceled)

39. An assay mixture comprising (a) reagents for measuring protein levels of Vascular Endothelial Growth Factor A (VEGFA), Transforming Growth Factor Beta 1 (TGFB1), and/or Colony Stimulating Factor 1 (CSF1) in a biological sample; and (b) a biological sample from a subject suffering from, or suspected of suffering from, nonalcoholic fatty liver disease (NAFLD).

40. The assay mixture of claim 39, wherein the assay mixture comprises (i) an antibody or antigen-binding fragment thereof that specifically binds to VEGFA, (ii) an antibody or antigen-binding fragment thereof that specifically binds to TGFB1; (iii) an antibody or antigen-binding fragment thereof that specifically binds to CSF1; or (iv) any combination of (i) to (iii).

41-45. (canceled)
